(19) United States
(12) Patent Application Publication
(10) Pub. No.: US 2024/0254530 A1
(43) Pub. Date: Aug. 1, 2024

(54) NON-ANIMAL CHONDROITIN SULFATE OLIGOSACCHARIDE AND PREPARATION METHOD THEREOF

(71) Applicant: JIANGNAN UNIVERSITY, Wuxi (CN)

(72) Inventors: Shuqin XU, Wuxi (CN); Jinghua CHEN, Wuxi (CN); Zhuqun WANG, Wuxi (CN)

(21) Appl. No.: 18/584,708

(22) Filed: Feb. 22, 2024

Related U.S. Application Data
(63) Continuation of application No. PCT/CN2022/114489, filed on Aug. 24, 2022.

(30) Foreign Application Priority Data
Aug. 25, 2021 (CN) 202110983725.7

(51) Int. Cl. C12P 19/26; C07H 11/00; C12N 5/079; C12P 19/18

(52) U.S. Cl. CPC C12P 19/26; C07H 11/00; C12N 5/0622; C12P 19/18; C12N 2506/08

(57) ABSTRACT

The invention provides a non-animal chondroitin sulfate oligosaccharide and a preparation method thereof. The method includes a one-step chemical procedure and an enzyme catalysis procedure that are orderly carried out, including: extracting Escherichia coli K4 polysaccharide, chemically removing the fructosyl group therefrom, and degrading with a chondroitin sulfate degrading enzyme to obtain a chondroitin oligosaccharide mixture; preparing chondroitin disaccharide to octasaccharide by separation by separation by a centrifugal ultrafiltration tube, Bio-Gel P-2 gel exclusion chromatography, and high performance liquid chromatography (HPLC); and enzymatically modifying the obtained products by 4-O-sulfation and 6-O-sulfation, to obtain chondroitin sulfate CS-A and CS-C oligosaccharides respectively. The raw materials are from non-animal sources, the pollution risk is low, the reaction conditions are mild and efficient, and the structures and molecular weights of the prepared chondroitin sulfate disaccharide to octasaccharide are definite, providing possibility for the research of chondroitin sulfate oligosaccharides with single degrees of polymerization.

This application is a Continuation Application of PCT/CN2022/114489, filed on Aug. 24, 2022, which claims priority to Chinese Patent Application No. 202110983725.7, filed on Aug. 25, 2021, which is incorporated by reference for all purposes as if fully set forth herein.

### FIELD OF THE INVENTION

The present invention relates to the technical field of biomedicines, and particularly to a non-animal chondroitin sulfate oligosaccharide and a preparation method thereof.

### DESCRIPTION OF THE RELATED ART

Chondroitin sulfate (CS) is an acidic mucopolysaccharide widely existing in human and animal cartilage, which is one of glycosaminoglycans. It has a structure formed by forming a disaccharide unit with glucuronic acid (Glc A) and N-acetylgalactosamine (GalNAc) through β-1,3 glycosidic bonds, and linking repeated disaccharide units by β-1, 4 glycosidic bonds. Substitutions with sulfate groups occur at different sites on the saccharide unit of CS, forming various chondroitin sulfates, including CS-A, CS-B, CS-C, CS-D, and CS-E. CS has high biological activity, for example, in inhibiting or promoting axon growth and regeneration, participating in inflammatory reaction and promoting osteogenesis. Numerous studies show that compared with complex CS polysaccharides, CS oligosaccharides have relatively uniform degree of polymerization and structure, which greatly avoids the ambiguity of polysaccharides' mechanism of action in particular biological activities; and have small molecular weights and is easy to absorb. Therefore, oligosaccharide fragments with controllable structure and molecular weight can be prepared to study their biological activities.

CS-A and CS-C are prepared by Zhou ZhengXiong and others by using a two-step biological strategy, in which the fermentation production of CH by recombinant Bacillus subtilis is optimized, and then CS-A and CS-C are synthesized with CH produced by catalytic fermentation in a sulfation conversion system having aryl sulfotransferase IV (ASST IV), chondroitin 4-O-sulfotransferase (C4ST), and chondroitin 6-O-sulfotransferase (C6ST) in combination, with a conversion rate of 98% and 96% respectively. However, regardless of direct fermentation to produce CS or indirect fermentation to produce CH and then sulfation into CS, there are bottlenecks in product purification and identification. There is no report on the production of CH or CS with clear structure by fermentation.

Application No. 201711216846.9 discloses a method for preparing chondroitin sulfate D tetrasaccharide, which includes the steps of alcohol precipitation, separation by anion exchange chromatography, separation by gel chromatography and others. Application No. 201210159448.9 discloses a method for preparing oligosaccharides with different molecular weights, which includes enzymolysis, molecular sieve, ion exchange and preparative electrophoresis. All of the above means are used for the efficient purification of highly sulfated animal chondroitin sulfate, such as chondroitin sulfate D or chondroitin sulfate E oligosaccharide.

At present, the separation and preparation methods of non-animal chondroitin sulfate oligosaccharides with specific structures and molecular weights have not been reported.

### SUMMARY OF THE INVENTION

To solve the above technical problems, the present invention provides a non-animal chondroitin sulfate oligosaccharide and a preparation method thereof.

A non-animal chondroitin sulfate oligosaccharide has a structural formula below:

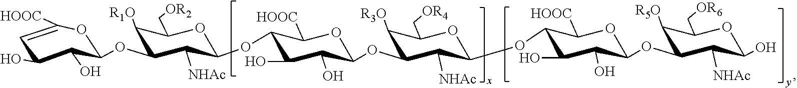

where R₁, R₂, R₃, R₄, R₅, and R₆ are independently selected from —H or —SO₃H;
x is an integer from 0 to 2; and
y is 0 or 1.

A method for preparing a non-animal chondroitin sulfate oligosaccharide includes the following steps:
(1) chemically removing the fructosyl group that is β-linked at position 3 of glucuronic acid in K4 polysaccharide, to obtain DK4;
(2) degrading DK4 obtained in Step (1) with a chondroitin sulfate degrading enzyme, to obtain a chondroitin oligosaccharide mixture; and
(3) subjecting the chondroitin oligosaccharide mixture obtained in Step (2) to ultrafiltration and centrifugation and chromatographic separation to obtain a chondroitin oligosaccharide, modifying the chondroitin oligosaccharide by sulfation with a chondroitin sulfate sulfotransferase, such that the N-acetylgalactosamine (GalNAc) in the chondroitin oligosaccharides is modified by 4-O-sulfation, 6-O-sulfation, or 4-O-sulfation and then 6-O-sulfation, to obtain the non-animal chondroitin sulfate oligosaccharide. The chondroitin sulfate sulfotransferase is one or more selected from chondroitin sulfate 4-O-sulfotransferase (CS4OST), chondroitin sulfate 6-O-sulfotransferase (CS6OST) and 4-O-sulfation-GalNAc-4-O-sulfotransferase (GalNAc4S-4OST).

In an embodiment of the present invention, in Step (1), the chemical removal comprises: dissolving the K4 polysaccharide in a 0.01-0.1 mol/L acid solution, heating to remove the fructosyl residue from glucuronic acid (GlcA) in K4 polysaccharide, cooling to room temperature, dialyzing, and freeze-drying under vacuum to obtain DK4. The acid is selected from hydrochloric acid, acetic acid, sulfuric acid or trifluoroacetic acid.

In an embodiment of the present invention, the mass to volume ratio of the K4 polysaccharide to the trifluoroacetic acid solution is 10:1-15:1 (m/v).

In an embodiment of the present invention, in Step (2), the chondroitin sulfate degrading enzyme is chondroitin sulfate degrading enzyme ChAC, chondroitin sulfate degrading enzyme ChABC or hyaluronidase.

In an embodiment of the present invention, the purification of the chondroitin oligosaccharide mixture comprise the following steps: (1) subjecting the reaction solution to 30 kDa, 10 kDa, 3 kDa, and 1 kDa ultrafiltration and centrifugation sequentially, and collecting the supernatant; (2) separating the supernatant by chromatography on Bio-Gel P-2 gel, and collecting the eluate; and (3) collecting the eluate, detecting by HPLC, and collecting the analyte at a wavelength of 232 nm, to obtain the non-animal chondroitin oligosaccharide of single component.

In an embodiment of the present invention, in Step (2), the degradation comprises the following steps: dissolving DK4 in an enzymolysis buffer; adding the chondroitin sulfate degrading enzyme; and reacting at 25-37° C. for 10 min-24 hrs; and after the reaction, heating, undergoing solid-liquid separation, and collecting the filtrate, to obtain the chondroitin oligosaccharide mixture.

In an embodiment of the present invention, the enzymolysis buffer for the chondroitin sulfate degrading enzyme ChAC comprises 15-25 mM Tris-HCl aqueous solution, and has a pH value of 7.0-7.5; the enzymolysis buffer for the chondroitin sulfate degrading enzyme ChABC comprises 80-120 mM Tris and 120-150 mM sodium acetate aqueous solution, and has a pH value of 7.5-8.0.

In an embodiment of the present invention, the weight ratio of the chondroitin sulfate degrading enzyme to DK4 is 0.2:12-1:12; and the enzymolysis time is 10 min-24 hrs.

In an embodiment of the present invention, in Step (3), the chondroitin oligosaccharide, the chondroitin sulfate sulfotransferase, and the sulfate donor 3′-phosphoadenosine-5′-phosphosulfate (PAPS) are mixed and reacted in a buffer, and the reaction solution is purified after the reaction, to obtain the non-animal chondroitin sulfate oligosaccharide.

In an embodiment of the present invention, the buffer has a composition comprising: 0.8-1.2 mol/L MOPS, pH 7.0-7.5, 200-300 mmol/L MnCl₂, and water as the solvent.

In an embodiment of the present invention, the molar ratio of the sulfate donor PAPS to the chondroitin oligosaccharide is 4:1-0.5:1.

In an embodiment of the present invention, the molar ratio of the chondroitin oligosaccharide to the chondroitin sulfate sulfotransferase is 1:0.1-1:20.

In an embodiment of the present invention, the mobile phase in Step (2) is a 0.05-0.1 mol/L ammonium bicarbonate aqueous solution; and the flow rate of the mobile phase is 0.125-0.167 mL/min.

The non-animal chondroitin sulfate oligosaccharide is useful in the preparation of drugs for treating nervous system diseases.

In an embodiment of the present invention, the non-animal chondroitin sulfate oligosaccharide is useful in the in-vitro differentiation of oligodendrocyte precursor cells, particularly, in the induction of oligodendrocyte precursor cells to differentiate into oligodendrocytes.

In an embodiment of the present invention, the oligodendrocyte precursor cells are cultured in a medium containing a non-animal chondroitin sulfate oligosaccharide.

In an embodiment of the present invention, the culture medium is a DMEM/F12 cell culture medium containing B27, Glutamax and P/S.

Preferably, the culture medium has a composition comprising: 96% DMEM/F12 cell culture medium, 2% B27, 1% Glutamax additive, and 1% P/S (penicillin and streptomycin).

The oligodendrocyte precursor cells, also known as NG2 glial cells, are self-renewing cells with multidirectional differentiation potential. To rule out ethical issues, when applied in human body, primary murine oligodendrocyte precursor cells are (OPC) after ethical review and approval are used in the present invention, including commercial oligodendrocyte precursor cells (for example, mouse oligodendrocyte precursor cell (HTX2431), human oligodendrocyte precursor cell (HTX2155), and rat oligodendrocyte precursor cell lines (OLN-93, R1600, and CG4). It is to be noted that oligodendrocyte precursor cells used in the present invention cannot develop into complete individuals.

Compared with the prior art, the technical solution of the present invention has the following advantages:

In the present invention, the K4 polysaccharide from which the fructosyl group is chemically removed is used as a substrate. The substrate is degraded by using the chondroitin sulfate degrading enzymes ChAC and ChABC to obtain a chondroitin oligosaccharide mixture. After separation by ultrafiltration and centrifugation, Bio-Gel P-2 gel exclusion chromatography, and HPLC, chondroitin oligosaccharides with definite structures and molecular weights are obtained, which are mainly disaccharide and tetrasaccharide, and have a molecular weight of 379 Da and 758 Da respectively. The chondroitin disaccharide accounts for 31%-48%, and the chondroitin tetrasaccharide accounts for 27%-55%. The resulting products are respectively enzymatically modified by 4-O-sulfation and 6-O-sulfation, to obtain chondroitin sulfate CS-A and CS-C oligosaccharides. The present invention relates to a non-animal chondroitin sulfate oligosaccharide and a preparation method thereof. The raw materials of this method are from non-animal sources, have stable structures, low pollution and high preparation efficiency. The structures and molecular weights of the prepared chondroitin sulfate oligosaccharides are definite, providing possibility for the research of chondroitin sulfate oligosaccharides with single degrees of polymerization.

In the present invention, oligosaccharides of even number of chondroitin prepared by enzymatic degradation are used as a substrate, where the oligosaccharide has a non-reducing end of unsaturated glycuronic acid (ΔHexUA), a reducing end of N-acetylgalactosamine (GalNAc), and a repeating unit of GlcA-GalNAc disaccharide. Based on enzymatic synthesis, GalNAc on the chondroitin oligosaccharide chain is modified by 4-O-sulfation using chondroitin sulfate 4-O-sulfotransferase (CS4OST) and the sulfate donor PAPS, to obtain a structure where the non-reducing terminal disaccharide GalNAc is modified by 4-O-sulfation, that is, ΔHexUA-GalNAc4S. The catalytic activity of CS4OST for the chondroitin oligosaccharide proceeds from the non-reducing end to the reducing end of the sugar chain sequentially. Therefore, a CS-A subtype with continuous sulfated pattern can be artificially synthesized by this method. By adding PAPS and CS4OST, to obtain fully sulfated CS-A oligosaccharide. 4-hydroxyl groups on all GalNAc of the chondroitin oligosaccharide are replaced by the sulfate groups. For animal-derived CS subtype oligosaccharides, because the natural CS is a mixture, the preparation of CS-subtype disaccharide and tetrasaccharide is mainly focused on at present, and it is difficult to obtain large fragments of fully sulfated CS oligosaccharides.

For chondroitin sulfate 6-O-sulfotransferase (CS6OST), the enzymatically catalytic characteristics are similar to those of CS4OST, including 6-O-sulfation modification of GalNAc on the non-reducing terminal disaccharide, and the catalytic activity proceeds from the non-reducing end to the reducing end of the sugar chain sequentially. Therefore, CS-C oligosaccharides with continuous sulfation pattern and full sulfation can be synthesized by this method.

CS-E is a subtype with less content in nature, and the commercial CS-E still contains 40% of other disaccharides. The CS-E oligosaccharide fragment with continuous sulfation pattern is mainly a fragment that is not larger than hexasaccharide. A large number of reports indicate that CS-E has a good ability to promote axonal regeneration, and the larger the CS-E fragment is, the more remarkable the effect will be. With above-synthesized CS-A oligosaccharide is used as a substrate, CS-E oligosaccharide with continuous sulfation pattern and full sulfation can be synthesized by using 4-O-sulfated GalNAc-6-O-sulfotransferase (GalNAc4S-6OST), in which the non-reducing terminal GalNAc has a disulfated structure.

### BRIEF DESCRIPTION OF THE DRAWINGS

To make the disclosure of the present invention more comprehensible, the present invention will be further described in detail by way of specific embodiments of the present invention with reference the accompanying drawings, in which:

FIG. 1 shows gradient elution of K4 polysaccharide by DEAE gel column, in which the horizontal ordinate is the elution volume and the vertical ordinate is the ultraviolet absorbency.

FIG. 2 is a ¹H NMR spectrum of K4 polysaccharide in Example 1 of the present invention.

FIG. 3 is a multi-angle laser light scattering analysis diagram of K4 polysaccharide in Example 1 of the present invention, in which the horizontal ordinate is the time, and the vertical ordinate is the response signal.

FIG. 4 is a ¹H NMR spectrum of DK4 polysaccharide in Example 2 of the present invention.

FIG. 5 is a multi-angle laser light scattering analysis diagram of DK4 in Example 2 of the present invention, in which the horizontal ordinate is the time, and the vertical ordinate is the response signal.

FIG. 6 is a Bio-Gel P-2 gel chromatogram of the fraction of less than 1 kDa obtained by passing the product obtained after 24 hrs of enzymolysis of DK4 by ChABC through an ultrafiltration tube in Example 3 of the present invention, in which the horizontal ordinate is the elution volume and the vertical ordinate is the absorbency at 232 nm.

FIG. 7 shows a HPLC chromatograph and a MS spectrum of a disaccharide prepared by enzymolysis of DK4 by ChABC in Example 3 of the present invention.

FIG. 8 is a Bio-Gel P-2 gel chromatogram of the fraction of less than 1 kDa obtained by passing the product obtained after 24 hrs of enzymolysis of DK4 by ChAC through an ultrafiltration tube in Example 4 of the present invention, in which the horizontal ordinate is the elution volume and the vertical ordinate is the absorbency at 232 nm.

FIG. 9 shows a HPLC chromatograph and a MS spectrum of a disaccharide prepared by enzymolysis of DK4 by ChAC in Example 4 of the present invention.

FIG. 10 is a Bio-Gel P-2 gel chromatogram of the fraction of 1 kDa −3 kDa obtained by passing the product obtained after 0.5 hr of enzymolysis of DK4 by ChABC through an ultrafiltration tube in Example 5 of the present invention, in which the horizontal ordinate is the elution volume and the vertical ordinate is the absorbency at 232 nm.

FIG. 11 shows a HPLC chromatograph and a MS spectrum of a tetrasaccharide prepared by enzymolysis of DK4 by ChABC in Example 5 of the present invention.

FIG. 12 shows a HPLC chromatograph and a MS spectrum of a hexasaccharide prepared by enzymolysis of DK4 by ChABC in Example 6 of the present invention.

FIG. 13 shows a HPLC chromatograph and a MS spectrum of an octasaccharide prepared by enzymolysis of DK4 by ChABC in Example 7 of the present invention.

FIG. 14 is an SDS-PAGE electrophoretogram of chondroitin sulfate 4-O-sulfotransferase in Example 8 of the present invention.

FIG. 15 shows a HPLC chromatograph and a MS spectrum of a CSA tetrasaccharide analogue in Example 8 of the present invention.

FIG. 16 is an SDS-PAGE electrophoretogram of chondroitin sulfate 6-O-sulfotransferase in Example 10 of the present invention.

FIG. 17 shows a HPLC chromatograph and a MS spectrum of a mono-sulfated CSC tetrasaccharide analogue in Example 10 of the present invention.

FIG. 18 shows a HPLC chromatograph and a MS spectrum of a disulfated CSC tetrasaccharide analogue in Example 11 of the present invention.

FIG. 19 (FIGS. 19A, 19B, and 19C) is a schematic diagram showing the preparation of non-animal CS-A, CS-C and CS-E oligosaccharides with K4 polysaccharide.

FIG. 20 is a laser confocal microscopy image showing the hexasaccharide-induced differentiation of OPCs into OLs.

FIG. 21 shows the proportion of OPCs induced to differentiate into OLs by tetrasaccharide, hexasaccharide, and octasaccharide.

FIG. 22 shows the proportion of OPCs induced to differentiate into OLs by CSA-tetrasaccharide, CSC-tetrasaccharide and CSE-tetrasaccharide.

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

### Example 1: Preparation of Escherichia coli K4 Polysaccharide

Escherichia coli K4 polysaccharide was prepared by culture in a 15 L fermentor. Seed culture medium: sodium chloride 10 g/L, tryptone 10 g/L, and yeast extract 5 g/L; Fermentation medium: glycerol 20 g/L, (NH₄)₂HPO₄ 4 g/L, MgSO₄·7 H₂O 1.4 g/L, citric acid 1.7 g/L, KH₂PO₄ 13 g/L, and trace element solution 10 mL/L; and Feed medium: glycerol 500 g/L, MgSO₄·7H₂O 20 g/L, and vitamin B 250 mg/L, adjusted to pH=7.

Fermentation conditions: inoculation amount 10%, temperature 37° C., pH 7, rotational speed 400-800 rpm, dissolved oxygen controlled to be less than 30% during fermentation, and culture time 48 hrs.

After the fermentation, the fermentation broth was centrifuged at 8000 rpm for 15 min at a low temperature, concentrated by boiling, precipitated with an alcohol, deproteinized with a savage reagents, dialyzed, rotary evaporated, and freeze-dried. The crude polysaccharide was collected, and the yield was 1.02 g/L.

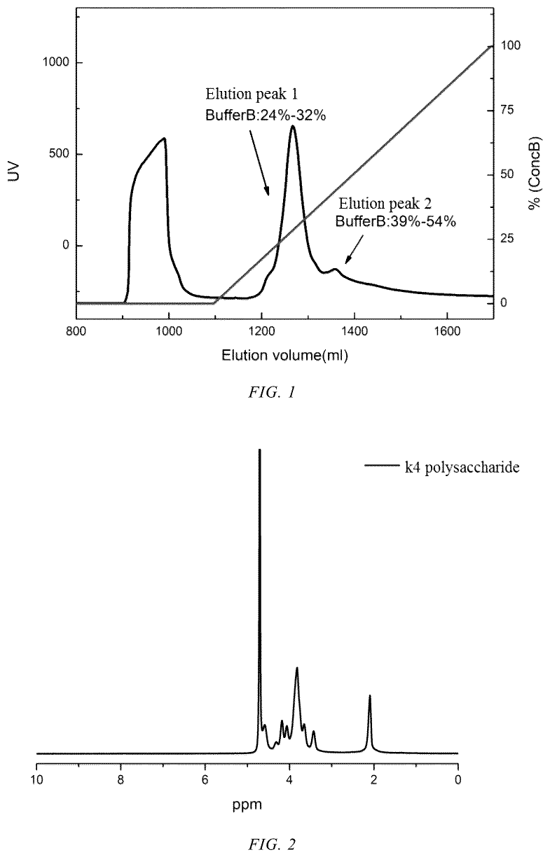

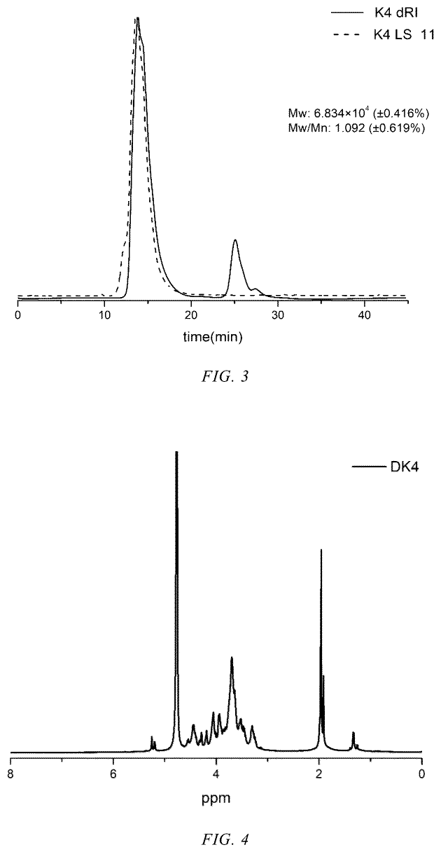

1 g of crude polysaccharide was purified by DEAE gel column chromatography, and Peak 1 of the target fraction was collected, as shown in FIG. 1. It was dialyzed in a dialysis bag with molecular weight cut-off of 3000 Da, concentrated by rotary evaporation, and freeze-dried to obtain pure K4 polysaccharide. The structure was confirmed by nuclear magnetic resonance, as shown in FIG. 2. The purity of the polysaccharide was determined and analyzed by gel permeation chromatography SEC-MALLS and the molecular weight was also determined, as shown in FIG. 3. Chromatographic conditions: Chromatographic column Shodex OHpak-SB 803, mobile phase 0.2 M NaCl, flow rate 0.5 mL/min, sample dissolved in the mobile phase at a concentration of 1 mg/mL, volume of injection 100 μL. The molecular weight is determined to be 68.3 kDa, and the polydispersity index PDI is 1.09. The results show that a polysaccharide with relatively uniform component could be prepared by the above methods.

### Example 2: Preparation of Chondroitin DK4

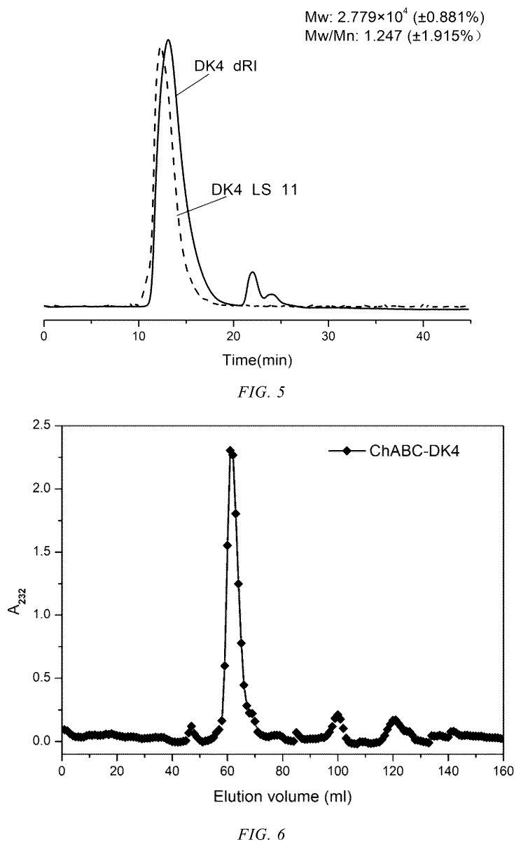

100 mg of pure K4 polysaccharide was added to 10 mL of 0.025 M trifluoroacetic acid (TFA), and reacted at 100° C. for 30 min. After the reaction, the solution was cooled to room temperature, dialyzed with distilled water in a dialysis bag with molecular weight cut-off of 3500 Da for three days, and freeze-dried to obtain DK4. The ¹H NMR spectrum of DK4 is shown in FIG. 4, which shows that the chemical structure of DK4 is chondroitin. The results of SEC-MALLS are shown in FIG. 5, showing that the molecular weight of DK4 is 27.7 kDa and the polydispersity index PDI is 1.24.

### Example 3: Enzymatic Preparation of Chondroitin Disaccharide by Using ChABC Enzyme

12 mg of purified chondroitin DK4 was weighed and dissolved in 6 mL of an enzymolysis buffer containing 100 mM Tris and 150 mM sodium acetate (pH 8.0), and allowed to stand in a constant temperature water bath at 37° C. for 10 min, to reach the enzymolysis reaction temperature. Then 1 mL of purified chondroitin sulfate degrading enzyme ChABC was added to the substrate solution, and reacted for 24 hrs in the constant temperature water bath. After the reaction, the metal bath was heated at 100° C. for 5 min to inactivate the enzyme, and the precipitate was removed by centrifuged at 8000 r/min for 10 min, to obtain a chondroitin oligosaccharide mixture.

The obtained chondroitin oligosaccharide mixture was passed through a centrifugal ultrafiltration tube of 30 kDa, 10 kDa, 3 kDa, and 1 kDa sequentially. After centrifugation at 4° C. and 4000 g for 30 min, the fractions were collected, which are mainly fractions of less than 1 kDa and 1-3 kDa. The fraction was frozen, centrifuged and concentrated to 200 μL, for use as the material for next separation.

The treated Bio-Gel P-2 packing was filled in a 1.6×80 cm glass chromatographic column, equilibrated overnight with deionized water, and then equilibrated with 2-3 column volumes of the mobile phase that is 0.1 mol/L ammonium bicarbonate solution. 200 μL of sample concentrated in the previous step was loaded, eluted with 5 column volumes of 0.1 M ammonium bicarbonate solution at a flow rate of 0.125 mL/min.

One tube (1 mL/tube) was collected every 5 min. The collected samples were detected at 232 nm by UV-VIS spectroscopy. The data was plotted to determine the peak position of the sugar. The curve of the fraction of less than 1 kDa after ultrafiltration is shown in FIG. 6.

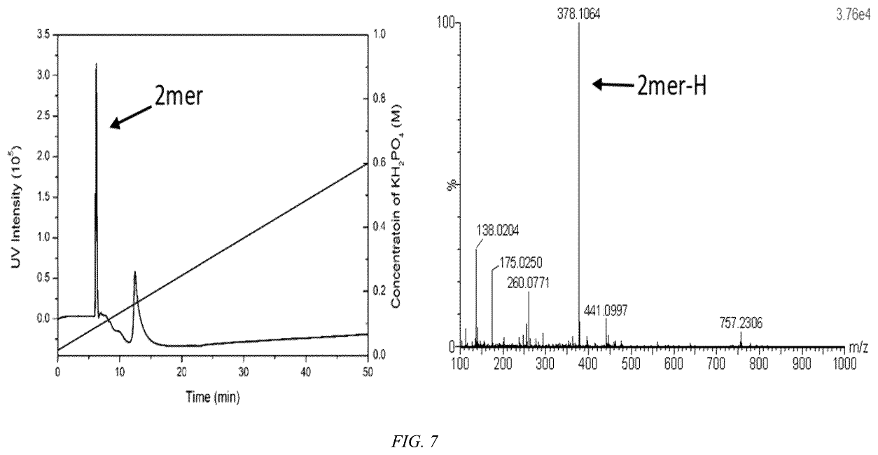

The reaction product at the largest peak was collected and concentrated to a certain volume. After the sample was filtered by a microporous filter membrane (0.22 μm), the purity was detected by HPLC. Detection condition: chromatographic column YMC-Pack polyamine II, mobile phase A: 16 mM KH₂PO₄, mobile phase B: 1 M KH₂PO₄, eluting over gradient with 0-60% B in 0-50 min, flow rate: 0.5 mL/min, and detection wavelength of UV detector: 232 nm. The samples were freeze-dried and detected by mass spectrometry, as shown in FIG. 7. The results show that the product obtained after degradation of DK4 by ChABC for 24 hrs mainly includes a disaccharide and a tetrasaccharide, having a molecular weight of 379 Da and 758 Da respectively. The fraction of less than 1 kDa is mainly disaccharide, with a yield of 31.6%. The fraction of 1-3 kDa is mainly tetrasaccharide, with a yield of 27.6%. The total yield of disaccharide and tetrasaccharide is up to 59.8%.

### Example 4: Enzymatic Preparation of Chondroitin Disaccharide by Using ChAC Enzyme

12 mg of purified DK4 was weighed and dissolved in 6 mL of an enzymolysis buffer containing 20 mM Tris-HCl (pH 7.0), and allowed to stand in a constant temperature water bath at 37ºC for 10 min, to reach the enzymolysis reaction temperature. Then 1 mL of purified chondroitin sulfate degrading enzyme ChAC was added to the substrate solution, and reacted for 24 hrs in the constant temperature water bath. After the reaction, the metal bath was heated at 100° C. for 5 min to inactivate the enzyme, and the precipitate was removed by centrifuged at 8000 r/min for 10 min, to obtain a chondroitin oligosaccharide mixture.

The obtained chondroitin oligosaccharide mixture was passed through a centrifugal ultrafiltration tube of 1 kDa. After centrifugation at 4° C. and 4000 g for 30 min, the fractions were collected, which are mainly fractions of less than 1 kDa and 1-3 kDa. The fraction was frozen, centrifuged and concentrated to 200 μL, for use as the material for next separation.

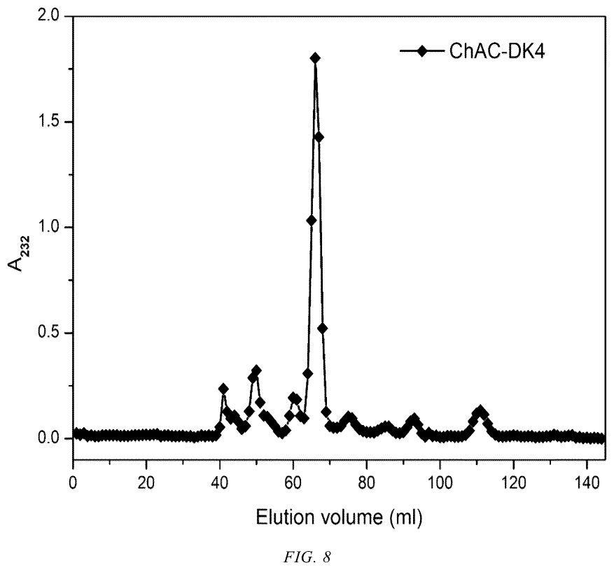

The treated Bio-Gel P-2 packing was filled in a 1.6×80 cm glass chromatographic column, equilibrated overnight with deionized water, and then equilibrated with 2-3 column volumes of the mobile phase that is 0.1 M ammonium bicarbonate solution. 200 μL of sample concentrated in the previous step was loaded, eluted with 5 column volumes of 0.1 M ammonium bicarbonate solution at a flow rate of 0.125 mL/min. One tube (1 mL/tube) was collected every 5 min. The collected samples were detected at 232 nm by UV-VIS spectroscopy. The data was plotted to determine the peak position of the sugar. The curve of the fraction of less than 1 kDa after ultrafiltration is shown in FIG. 8.

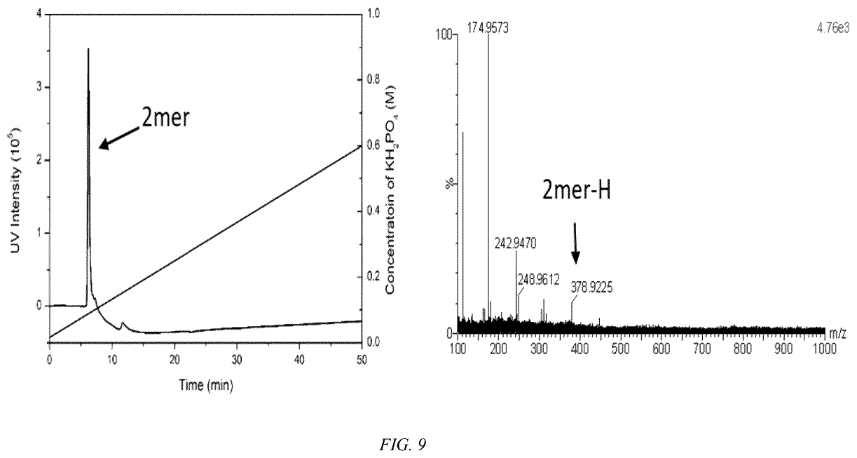

The reaction product at the largest peak was collected and concentrated to a certain volume. After the sample was filtered by a microporous filter membrane (0.22 μm), the purity was detected by HPLC. Detection and collection conditions: chromatographic column YMC-Pack polyamine II, mobile phase A: 16 mM KH₂PO₄, mobile phase B: 1 M KH₂PO₄, eluting over gradient with 0-60% B in 0-50 min, flow rate 0.5 mL/min, and detection wavelength of UV detector: 232 nm. The samples were freeze-dried and detected by mass spectrometry, as shown in FIG. 9. The results show that the product obtained after degradation of DK4 by ChAC for 24 hrs mainly includes a disaccharide and a tetrasaccharide, having a molecular weight of 379 Da and 758 Da respectively. The yield of the two can be up to 60.8%. The disaccharide is mainly present in the fraction of less than 1 kDa, and the yield is up to 31.9%.

### Example 5: Enzymatic Preparation of Chondroitin Tetrasaccharide by Using ChABC Enzyme

12 mg of purified chondroitin DK4 was weighed and dissolved in 6 mL of an enzymolysis buffer containing 100 mM Tris and 150 mM sodium acetate (pH 8.0), and allowed to stand in a constant temperature water bath at 37° C. for 10 min, to reach the enzymolysis reaction temperature. Then 1 mL of purified chondroitin sulfate degrading enzyme ChABC was added to the substrate solution, and reacted for 0.5 hrs in the constant temperature water bath. After the reaction, the metal bath was heated at 100° C. for 5 min to inactivate the enzyme, and the precipitate was removed by centrifuged at 8000 r/min for 10 min, to obtain a chondroitin oligosaccharide mixture.

The obtained chondroitin oligosaccharide mixture was passed through a centrifugal ultrafiltration tube of 30 kDa, 10 kDa, and 3 kDa, and 1 kDa sequentially. After centrifugation at 4° C. and 4000 g for 30 min, the fractions were collected, which are mainly fractions of 1-3 kDa and 3-10 kDa. The fraction was frozen, centrifuged and concentrated to 200 μL, for use as the material for next separation.

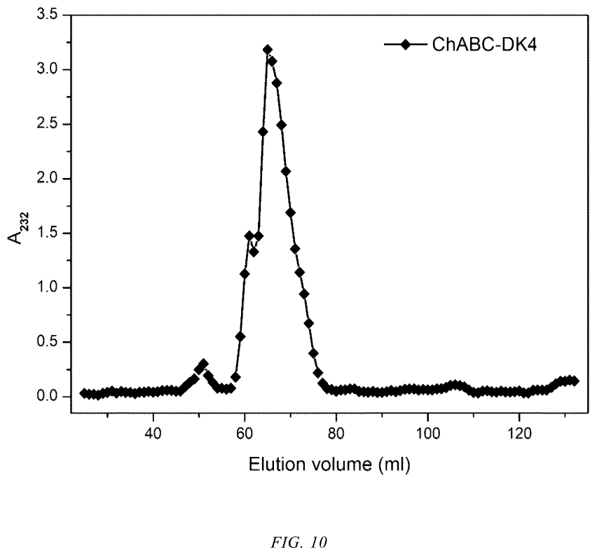

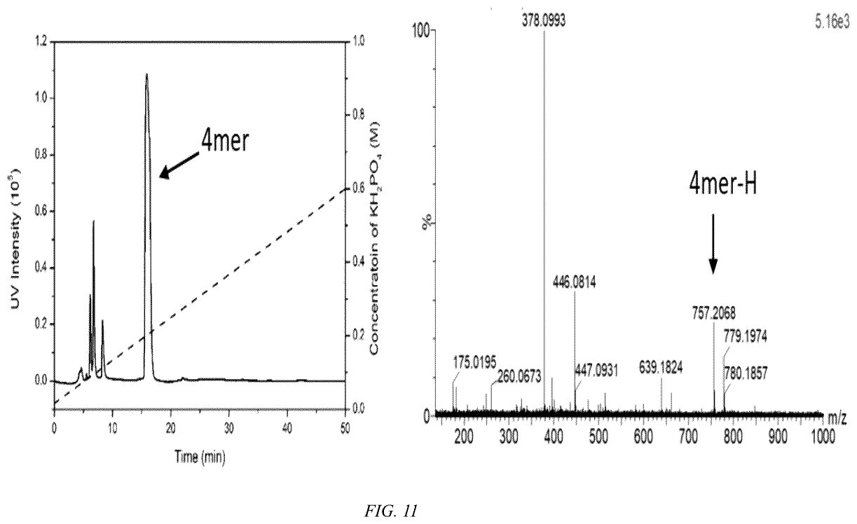

The treated Bio-Gel P-2 packing was filled in a 1.6×80 cm glass chromatographic column, equilibrated overnight with deionized water, and then equilibrated with 2-3 column volumes of the mobile phase that is 0.1 M ammonium bicarbonate solution. 200 μL of sample concentrated in the previous step was loaded, eluted with 5 column volumes of 0.1 M ammonium bicarbonate solution at a flow rate of 0.167 mL/min. One tube (1 mL/tube) was collected every 6 min. The collected samples were detected at 232 nm by UV-VIS spectroscopy. The data was plotted to determine the peak position of the sugar. The curve of the fraction of 1-3 kDa after ultrafiltration is shown in FIG. 10.

The reaction product at the largest peak was collected and concentrated to a certain volume. After the sample was filtered by a microporous filter membrane (0.22 μm), the purity was detected by HPLC. Detection and collection conditions: chromatographic column YMC-Pack polyamine II, mobile phase A: 16 mM KH₂PO₄, mobile phase B: 1 M KH₂PO₄, eluting over gradient with 0-60% B in 0-50 min, the flow rate was 0.5 mL/min, and detection wavelength of UV detector: 232 nm. The results show that the product obtained after degradation of DK4 by ChABC for 0.5 hrs mainly includes a disaccharide and a tetrasaccharide, having a molecular weight of 379 Da and 758 Da respectively. The fractions of 1-3 kDa and 3-10 kDa are both mainly tetrasaccharide. The liquid chromatography and mass spectrometry of tetrasaccharide are shown in FIG. 11. The yield of the tetrasaccharide can be up to 55.6%.

### Example 6: Enzymatic Preparation of Chondroitin Hexasaccharide by Using ChABC Enzyme

12 mg of purified chondroitin DK4 was weighed and dissolved in 6 mL of an enzymolysis buffer containing 100 mM Tris and 150 mM sodium acetate (pH 8.0), and allowed to stand in a constant temperature water bath at 37ºC for 10 min, to reach the enzymolysis reaction temperature. Then 0.2 mL of purified chondroitin sulfate degrading enzyme ChABC was added to the substrate solution, and reacted for 15 min in the constant temperature water bath. After the reaction, the metal bath was heated at 100° ° C. for 5 min to inactivate the enzyme, and the precipitate was removed by centrifuged at 8000 r/min for 10 min, to obtain a chondroitin oligosaccharide mixture.

The obtained chondroitin oligosaccharide mixture was passed through a centrifugal ultrafiltration tube of 10 kDa, 3 kDa, and 1 kDa sequentially. After centrifugation at 4° C. and 4000 g for 30 min, the fractions were collected, which are mainly fractions of 1-3 kDa and 3-10 kDa. The fraction was frozen, centrifuged and concentrated to 200 μL, for use as the material for next separation.

The treated Bio-Gel P-2 packing was filled in a 1.6×80 cm glass chromatographic column, equilibrated overnight with deionized water, and then equilibrated with 2-3 column volumes of the mobile phase that is 0.1 M ammonium bicarbonate solution. 200 μL of sample concentrated in the previous step was loaded, eluted with 5 column volumes of 0.1 M ammonium bicarbonate solution at a flow rate of 0.167 mL/min. One tube (1 mL/tube) was collected every 6 min. The collected samples were detected at 232 nm by UV-VIS spectroscopy. The data was plotted to determine the peak position of the sugar.

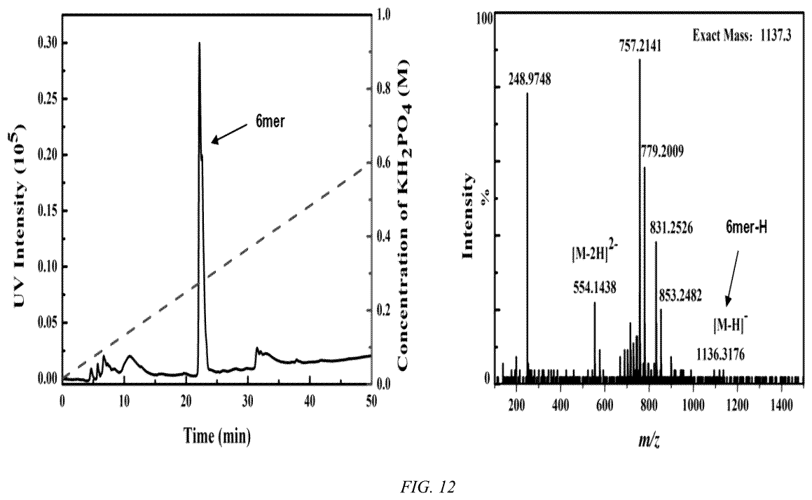

The reaction product at the largest peak was collected and concentrated to a certain volume. After the sample was filtered by a microporous filter membrane (0.22 μm), the purity was detected by HPLC. Detection and collection conditions: chromatographic column YMC-Pack polyamine II, mobile phase A: 16 mM KH₂PO₄, mobile phase B: 1 M KH₂PO₄, eluting over gradient with 0-60% B in 0-50 min, the flow rate was 0.5 mL/min, and detection wavelength of UV detector: 232 nm. The results show that the product obtained after degradation of DK4 by ChABC for 15 hrs mainly includes a tetrasaccharide and a hexasaccharide, having a molecular weight of 758 Da and 1137 Da respectively. The liquid chromatography and mass spectrometry of hexasaccharide are shown in FIG. 12. The yield of the hexasaccharide can be up to 26.2%.

### Example 7: Enzymatic Preparation of Chondroitin Octasaccharide by Using ChABC Enzyme

12 mg of purified chondroitin DK4 was weighed and dissolved in 6 mL of an enzymolysis buffer containing 100 mM Tris and 150 mM sodium acetate (pH 8.0), and allowed to stand in a constant temperature water bath at 37° C. for 10 min, to reach the enzymolysis reaction temperature. Then 0.2 mL of purified chondroitin sulfate degrading enzyme ChABC was added to the substrate solution, and reacted for 10 min in the constant temperature water bath. After the reaction, the metal bath was heated at 100° C. for 5 min to inactivate the enzyme, and the precipitate was removed by centrifuged at 8000 r/min for 10 min, to obtain a chondroitin oligosaccharide mixture.

The obtained chondroitin oligosaccharide mixture was passed through a centrifugal ultrafiltration tube of 10 and 1 kDa sequentially. After centrifugation at 4° C. and 4000 g for 30 min, the fraction was collected, which is mainly the fraction of 1-10 kDa. The fraction was frozen, centrifuged and concentrated to 200 μL, for use as the material for next separation.

The treated Bio-Gel P-2 packing was filled in a 1.6×80 cm glass chromatographic column, equilibrated overnight with deionized water, and then equilibrated with 2-3 column volumes of the mobile phase that is 0.1 M ammonium bicarbonate solution. 200 μL of sample concentrated in the previous step was loaded, eluted with 5 column volumes of 0.1 M ammonium bicarbonate solution at a flow rate of 0.167 mL/min. One tube (1 mL/tube) was collected every 6 min. The collected samples were detected at 232 nm by UV-VIS spectroscopy. The data was plotted to determine the peak position of the sugar.

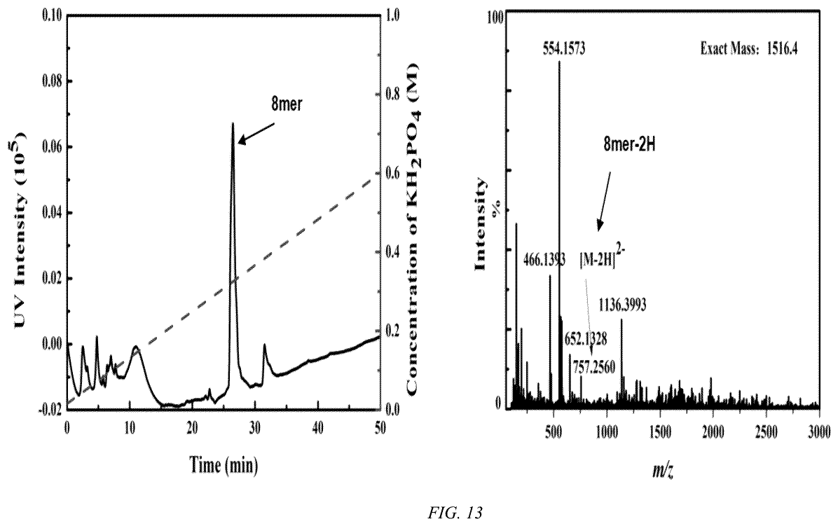

The reaction product at the largest peak was collected and concentrated to a certain volume. After the sample was filtered by a microporous filter membrane (0.22 μm), the purity was detected by HPLC. Detection and collection conditions: chromatographic column YMC-Pack polyamine II, mobile phase A: 16 mM KH₂PO₄, mobile phase B: 1 M KH₂PO₄, eluting over gradient with 0-60% B in 0-50 min, the flow rate was 0.5 mL/min, and detection wavelength of UV detector: 232 nm. The results show that the product obtained after degradation of DK4 by ChABC for 10 min mainly includes a tetrasaccharide, a hexasaccharide and an octasaccharide, having a molecular weight of 757 Da, 1137 Da and 1517 Da respectively. The liquid chromatography and mass spectrometry of octasaccharide are shown in FIG. 13. The yield of the octasaccharide can be up to 11.3%.

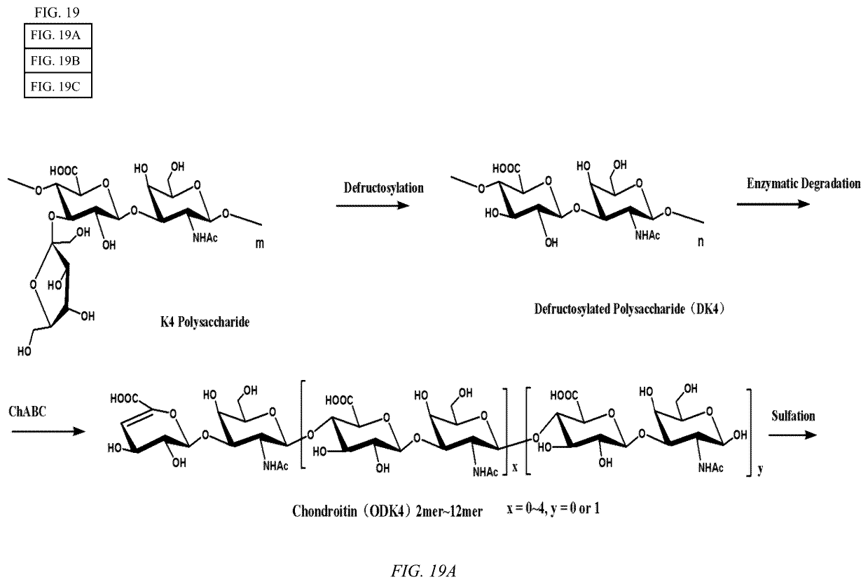

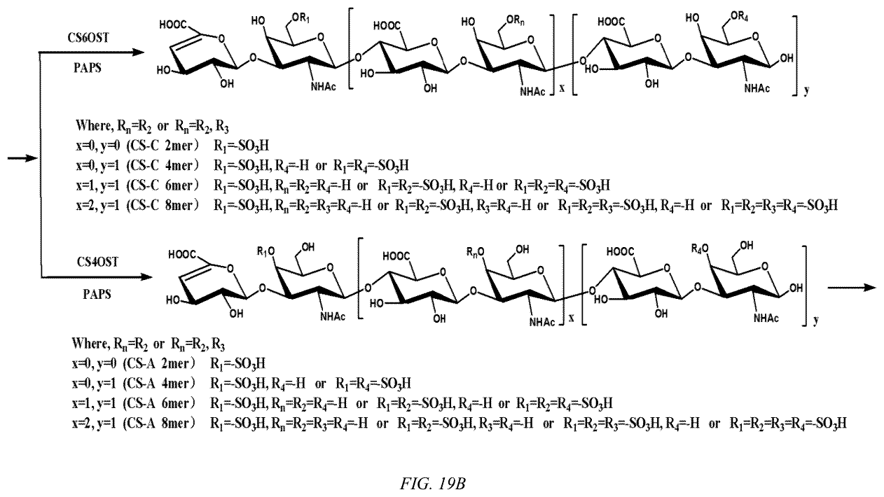

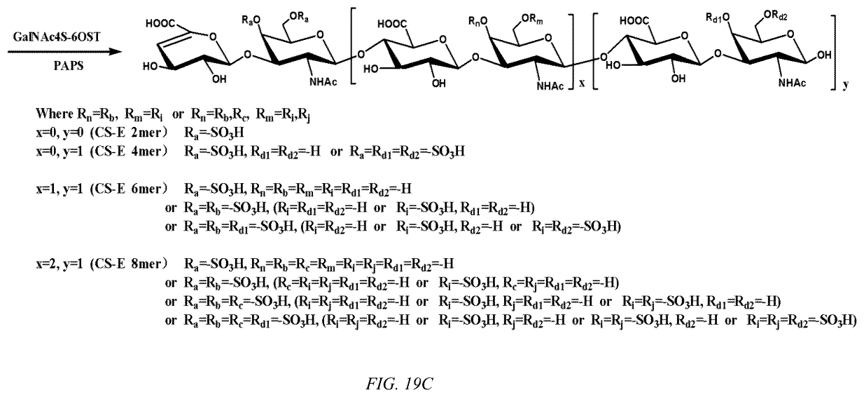

### Example 8: Synthesis of CS-A Tetrasaccharide 1 by Enzymatic 4-O-Sulfation Modification (Schematic Diagram is Shown in FIGS. 19A, 19B, and 19C)

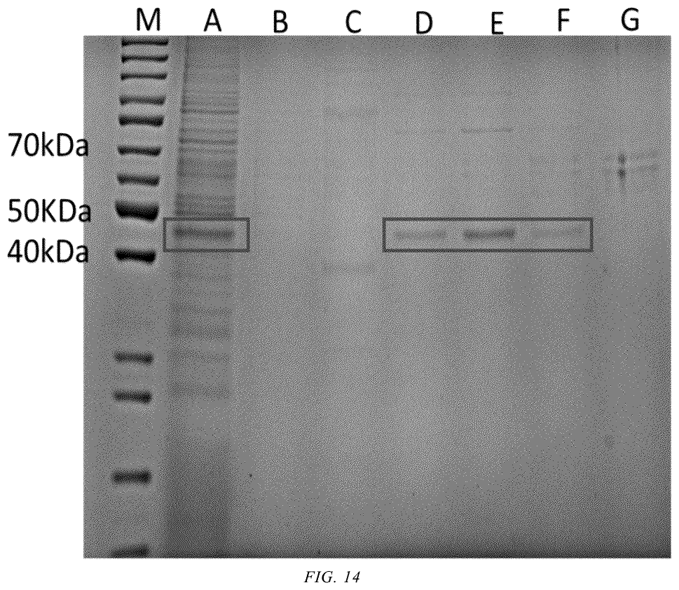

Sf-900™ III SFM medium was pre-warmed at room temperature for 20 min. The frozen Sf9 cells were removed from a liquid nitrogen tank, and immediately shaken quickly in a water bath at 37° ° C. The cells were transferred into a 10 ml centrifuge tube after they were completely thawed, and an appropriate amount of culture medium was added. After centrifugation at 800 rpm for 3 min, the supernatant was discarded. An appropriate amount of culture medium was added to dilute the Sf9 cells, and then the cells were transferred to a 125 mL shake flask and made up to 20-25 mL. The cells were cultured at 110 rpm and 27° C., and the medium was changed after 24 hrs. When the cell density reached 2×10⁶-6×10⁶ cell/mL and the living cell rate was 80-95%, subsequent cell passage and cell transfection were carried out. When the cell density was 12×10⁵-20×10⁵ cells/mL, the cells were infected with the recombinant virus of CS4OST (P3 generation of virus) and cultured in the dark at 27° C. for 3-4 days. After low-temperature centrifugation (8000 rpm, 15 min), the supernatant of the culture was collected, and filtered by a 0.22 μm filter membrane. The expressed protein CS4OST was purified by elution over gradient using a HisSep Ni-NTA 6FF His-tagged protein purification column. The expression of the protein was detected by SDS-PAGE, and the purity of the target protein was analyzed, as shown in FIG. 14.

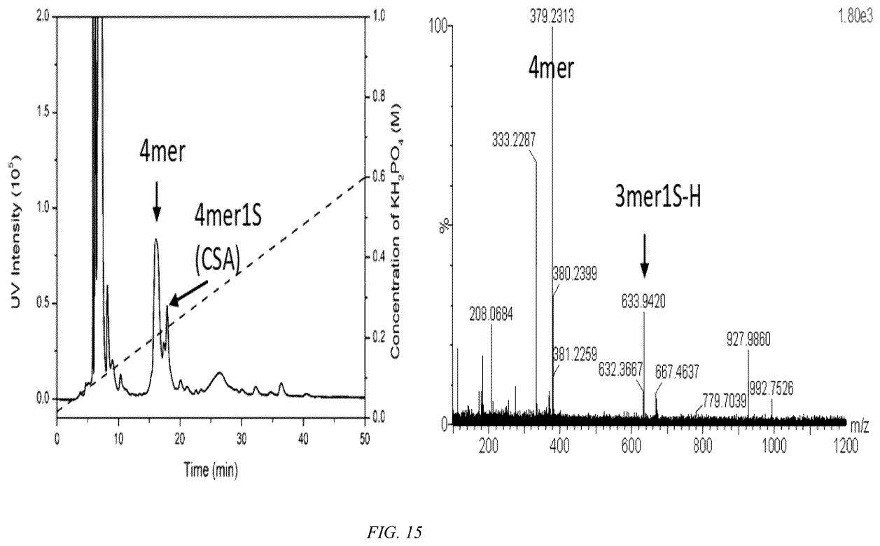

500 μg of chondroitin tetrasaccharide prepared in Example 5 was weighed and dissolved in 250 μL of ultrapure water, 50 μL of a buffer (1M MOPS, pH 7.0-7.5) and 50 μL of 200 mM MnCl₂ were added, and then 100 μL of the sulfate donor PAPS (about 1 mg/mL) and 550 μL of 4-O-sulfotransferase (4OST) having a molecular weight ranging from 40 kDa −50 kDa were added, and reacted on a shaker at 37° C. and 100 rpm for 12 hrs. The metal bath was heated at 100° C. for 5 min to terminate the reaction, and 112 μg of the main product that is mono-sulfated CS-A tetrasaccharide was obtained after centrifugation and filtration. As shown in FIG. 15, the peak of the product CS-A tetrasaccharide in the HPLC chromatogram appears at 18 min, and the peak of mono-sulfated trisaccharide fragment [3mer1S-H] appears at 633.9 in the MS spectrum. Due to the excessive substrate chondroitin tetrasaccharide, the reaction degree is limited by the amount of PAPS. According to the peak area of the product in the HPLC chromatogram, the sulfation conversion rate is 63%.

### Example 9: Synthesis of CS-A Tetrasaccharide 2 by Enzymatic 4-O-Sulfation Modification

100 μg of chondroitin tetrasaccharide prepared in Example 5 was weighed and dissolved in 50 μL of ultrapure water, 50 μL of a buffer (1M MOPS, pH 7.0-7.5) and 50 μL of 200 mM MnCl₂ were added, and then 300 μL of the sulfate donor PAPS (about 1 mg/mL) and 550 μL of 4-O-sulfotransferase (40ST) having a molecular weight ranging from 40 kDa −50 kDa were added, and reacted on a shaker at 37° C. and 100 rpm for 12 hrs. According to the reaction process, appropriate amount of the enzyme and the sulfate donor PAPS can be supplemented until the end of the reaction. The metal bath was heated at 100° ° C. for 5 min to terminate the reaction, and 98 μg of the main product that is disulfated CS-A tetrasaccharide was obtained after centrifugation and filtration. The peak of CS-A tetrasaccharide in HPLC chromatogram appears at 20 min, and the peak of disulfated tetrasaccharide [4mer2S-2H] appears at 458.1 in the MS spectrum. Due to the excessive PAPS and enzyme, the reaction is thorough. According to the peak area of the product in the HPLC chromatogram, the sulfation conversion rate is 81%.

### Example 10: Synthesis of CS-C Tetrasaccharide 1 by Enzymatic 6-O-Sulfation Modification (Schematic Diagram is Shown in FIGS. 19A, 19B, and 19C)

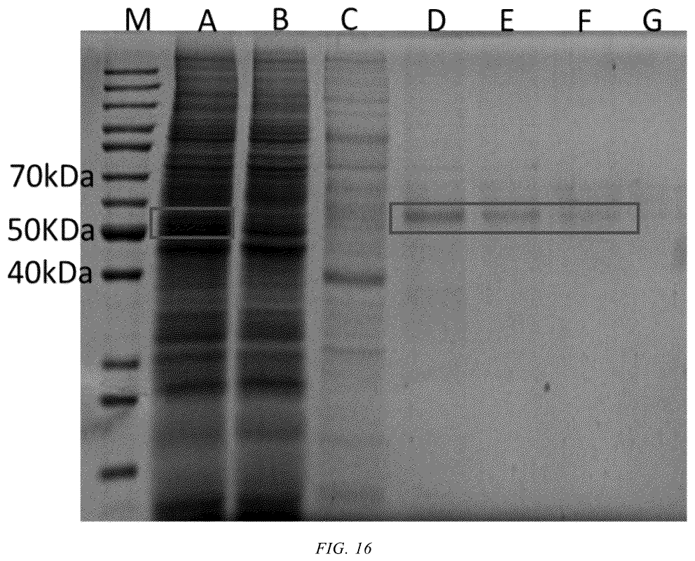

Sf-900™ III SFM medium was pre-warmed at room temperature for 20 min. The frozen Sf9 cells were removed from a liquid nitrogen tank, and immediately shaken quickly in a water bath at 37° C. The cells were transferred into a 10 ml centrifuge tube after they were completely thawed, and an appropriate amount of culture medium was added. 800 rpm, 3 min, the supernatant was discarded. An appropriate amount of culture medium was added to dilute the Sf9 cells, and then the cells were transferred to a 125 mL shake flask and made up to 20-25 ml. The cells were cultured at 110 rpm and 27° C., and the medium was changed after 24 hrs. When the cell density reached 2×10⁶-6×10⁶ cells/mL, and the living cell rate was 80-95%, subsequent cell passage and cell transfection were carried out. When the cell density was 12×10⁵-20×10⁵ cells/mL, the cells were infected with the recombinant virus of CS6OST (P3 generation of virus) and cultured in the dark at 27ºC for 3-4 days. After low-temperature centrifugation (8000 rpm, 15 min), the supernatant of the culture was collected, and filtered by a 0.22 μm filter membrane. The expressed protein CS6OST was purified by elution over gradient using a HisSep Ni-NTA 6FF His-tagged protein purification column. The expression of the protein was detected by SDS-PAGE, and the purity of the target protein was analyzed, as shown in FIG. 16.

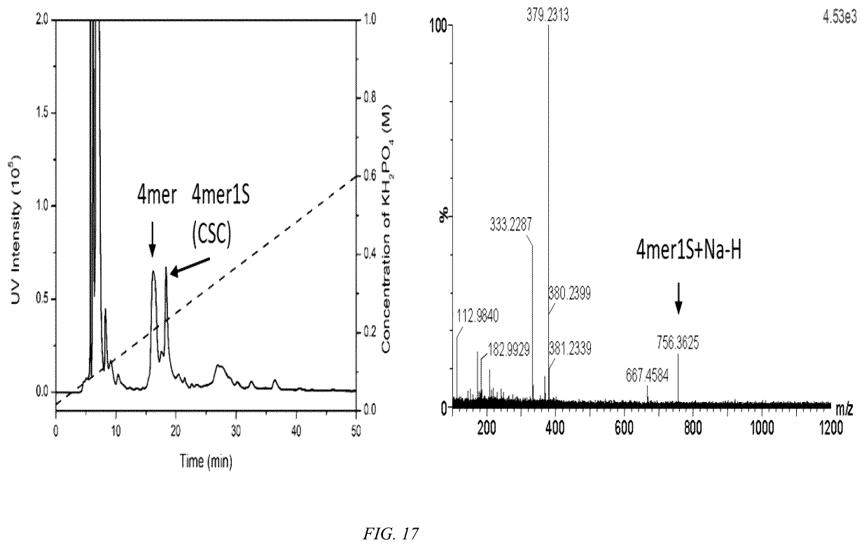

500 μg of chondroitin tetrasaccharide prepared in Example 5 was weighed and dissolved in 250 μL of ultrapure water, 50 μL of a buffer (1M MOPS, pH 7.0-7.5) and 50 μL of 200 mM MnCl₂ were added, and then 100 μL of the sulfate donor PAPS (about 1 mg/mL) and 550 μL of 6-O-sulfotransferase (6OST) having a molecular weight ranging from 50 kDa −60 kDa were added, and reacted on a shaker at 37° C. and 100 rpm for 12 hrs. The metal bath was heated at 100° ° C. for 5 min to terminate the reaction, and 174 μg of mono-sulfated CS-C tetrasaccharide was obtained after centrifugation and filtration. As shown in FIG. 17, the peak of the product CS-C tetrasaccharide in the HPLC chromatogram appears at 18 min, and the peak of mono-sulfated tetrasaccharide sodium salt [4mer1S+Na-H] appears at 860.3 in the MS spectrum. Due to the excessive substrate chondroitin tetrasaccharide, the reaction degree is limited by the amount of PAPS. According to the peak area of the product in the HPLC chromatogram, the sulfation conversion rate is 98%.

### Example 11: Synthesis of CS-C Tetrasaccharide 2 by Enzymatic 6-O-Sulfation Modification

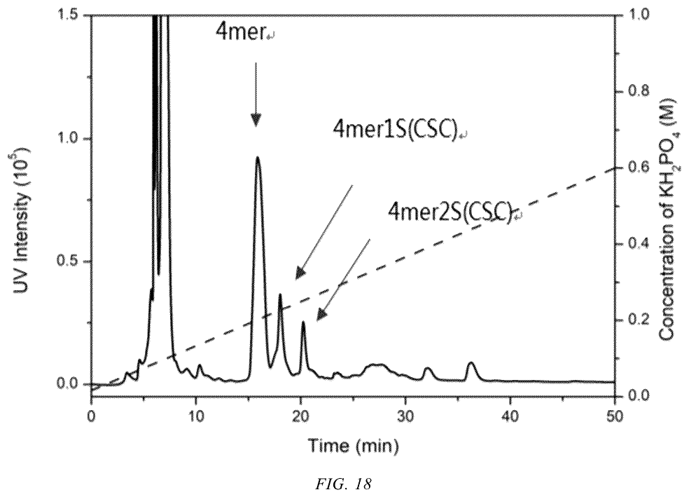

500 μg of chondroitin tetrasaccharide prepared in Example 5 was weighed and dissolved in 250 μL of ultrapure water, 50 μL of a buffer (1M MOPS, pH 7.0-7.5) and 50 μL of 200 mM MnCl₂ were added, and then 100 μL of the sulfate donor PAPS (about 1 mg/mL) and 550 μL of 6-O-sulfotransferase (6OST) having a molecular weight ranging from 50 kDa −60 kDa were added, and reacted on a shaker at 37° C. and 100 rpm for 12 hrs. According to the reaction process, appropriate amount of the enzyme and the sulfate donor PAPS can be supplemented until the end of the reaction. The metal bath was heated at 100° ° C. for 5 min to terminate the reaction, and 96.2 μg of mono-sulfated CS-C tetrasaccharide and 53.5 μg of disulfated CS-C tetrasaccharide were obtained after centrifugation and filtration. As shown in FIG. 18, the peak of the product mono-sulfated CS-C tetrasaccharide in the HPLC chromatogram appears at 18 min, and the peak of the disulfated tetrasaccharide appears at 20.2 min. In the MS spectrum, the peak of mono-sulfated tetrasaccharide sodium salt [4mer1S+Na-H] appears at 860.3 and the peak of disulfated tetrasaccharide [4mer2S-2H] appears at 458.1. Due to the excessive substrate chondroitin tetrasaccharide, the reaction degree is limited by the amount of PAPS. According to the peak area of the product in the HPLC chromatogram, the sulfation conversion rate is 86.5%.

### Example 12: Synthesis of CS-C Tetrasaccharide 3 by Enzymatic 6-O-Sulfation Modification

100 μg of chondroitin tetrasaccharide prepared in Example 5 was dissolved in 50 μL of ultrapure water, 50 μL of a buffer (1M MOPS, pH 7.0-7.5) and 50 μL of 200 mM MnCl₂ were added, and then 300 μL of the sulfate donor PAPS (about 1 mg/mL) and 550 μL of 6-O-sulfotransferase (6OST) were added, and reacted on a shaker at 37° ° C. and 100 rpm for 12 hrs. According to the reaction process, appropriate amount of the enzyme and the sulfate donor PAPS can be supplemented until the end of the reaction. The metal bath was heated at 100° C. for 5 min to terminate the reaction, and 111.6 μg of disulfated CS-C tetrasaccharide was obtained after centrifugation and filtration. The peak of the product disulfated CS-C tetrasaccharide in the HPLC chromatogram appears at 20.2 min, and the peak of disulfated tetrasaccharide sodium salt [4mer2S+Na-H] appears at 939.1 in the MS spectrum. According to the peak area of the product in the HPLC chromatogram, the sulfation conversion rate is 92.2%.

### Example 13: Synthesis of CS-E Tetrasaccharide by Enzymatic 4-O-Sulfation and 6-O-Sulfation Modification (Schematic Diagram is Shown in FIGS. 19A, 19B, and 19C)

100 μg of CS-A tetrasaccharide prepared in Example 7 was weighed and dissolved in 50 μL of ultrapure water, 50 μL of a buffer (1M MOPS, pH 7.0-7.5) and 50 μL of 200 mM MnCl₂ were added, and then 300 μL of the sulfate donor PAPS (about 1 mg/mL) and 550 μL of 4-O-sulfation-GalNAc- 6-O-sulfotransferase (GalNAc4S-6OST) were added, and reacted on a shaker at 37° C. and 100 rpm for 12 hrs. According to the reaction process, appropriate amount of the enzyme and the sulfate donor PAPS can be supplemented until the end of the reaction. The metal bath was heated at 100° C. for 5 min to terminate the reaction, and 67.8 μg of a disulfated CS-E tetrasaccharide analogue was obtained after centrifugation and filtration.

### Example 14: Ability of Chondroitin Oligosaccharide to Promote OPC Differentiation

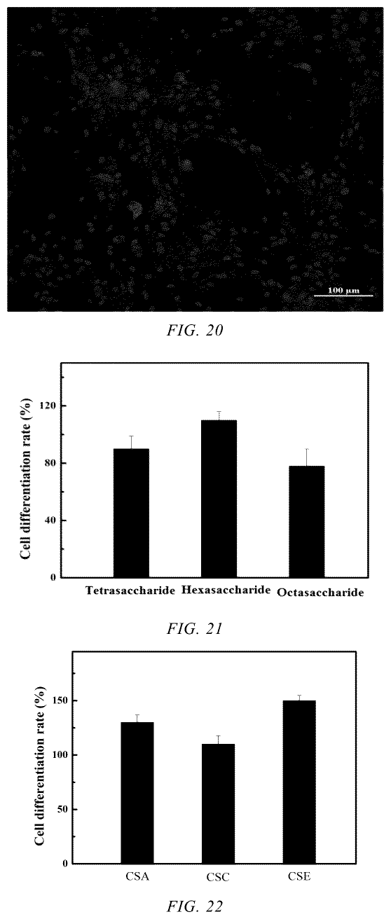

A mixed cell suspension of oligodendrocyte precursor cells (OPCs) and astrocytes (ASTs) was inoculated on a polylysine-coated confocal plate at 1×10⁵/mL. 1 mL of a differentiation medium containing chondroitin tetrasaccharide, hexasaccharide, and octasaccharide respectively and having a final concentration of 100 μg/mL (differentiation medium: 96% DMEM/F12 cell culture medium, 2% B27, 1% Glutamax additive, and 1% P/S) was added. The cells added with blank DMEM/F12 medium were used as a control. After being cultured for 24 hrs in a constant temperature incubator at 37° ° C. and 5% CO₂, the proportion of OPC differentiated into oligodendrocytes (OL) was investigated by immunofluorescence staining. The cells were immobilized with 4% paraformaldehyde solution, incubated with OL labeling rabbit polyclonal antibody (primary antibody) targeting myelin basic protein (MPB) and goat anti-rabbit antibody (secondary antibody) labeled with red fluorescent Cy3, washed with PBS, and imaged by laser confocal microscopy. The results are shown in FIGS. 20 and 21.

### Example 15: Ability of Chondroitin Sulfate Oligosaccharide to Promote OPC Differentiation

A mixed cell suspension of oligodendrocyte precursor cells (OPCs) and astrocytes (ASTs) was inoculated on a polylysine-coated confocal plate at 1×10⁵/mL, and 1 mL of a differentiation medium containing CS-A tetrasaccharide (prepared in Example 9), CS-C tetrasaccharide (prepared in Example 11) and CS-E tetrasaccharide (prepared in Example 13) respectively and having a final concentration of 100 μg/mL (differentiation medium: 96% DMEM/F12 cell culture medium, 2% B27, 1% Glutamax additive, and 1% P/S) was added. The cells added with blank DMEM/F12 medium were used as a control. After being cultured for 24 hrs in a constant temperature incubator at 37° C. and 5% CO₂, the proportion of OPC differentiated into oligodendrocytes (OLs) was investigated by immunofluorescence staining. The cells were immobilized with 4% paraformaldehyde solution, incubated with OL labeling rabbit polyclonal antibody (primary antibody) targeting myelin basic protein (MPB) and goat anti-rabbit antibody (secondary antibody) labeled with red fluorescent Cy3, washed with PBS, and imaged by laser confocal microscopy. The results are shown in FIG. 22.

Apparently, the above-described embodiments are merely examples provided for clarity of description, and are not intended to limit the implementations of the present invention. Other variations or changes can be made by those skilled in the art based on the above description. The embodiments are not exhaustive herein. Obvious variations or changes derived therefrom also fall within the protection scope of the present invention.

What is claimed is:

1. A non-animal chondroitin sulfate oligosaccharide, having a structural formula below: 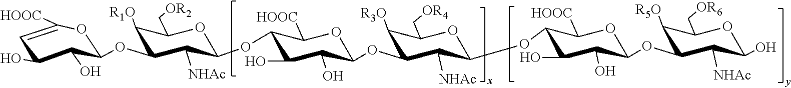
wherein R₁, R₂, R₃, R₄, R₅, and R₆ are independently selected from —H or —SO₃H;
x is an integer from 0 to 2; and
y is 0 or 1.

2. A method for preparing a non-animal chondroitin sulfate oligosaccharide according to claim 1, comprising the following steps:
(1) chemically removing the fructosyl group that is β-linked at position 3 of glucuronic acid in K4 polysaccharide, to obtain DK4;
(2) degrading DK4 obtained in Step (1) with a chondroitin sulfate degrading enzyme, to obtain a chondroitin oligosaccharide mixture;
(3) separating and purifying the chondroitin oligosaccharide mixture obtained in Step (2) to obtain a chondroitin oligosaccharide, modifying the chondroitin oligosaccharide by sulfation with a chondroitin sulfate sulfotransferase, such that the N-acetylgalactosamine in the chondroitin oligosaccharide is modified by 4-O-sulfation, 6-O-sulfation, or 4-O-sulfation and then 6-O-sulfation, to obtain the non-animal chondroitin sulfate oligosaccharide, wherein the chondroitin sulfate sulfotransferase is one or more selected from chondroitin sulfate 4-O-sulfotransferase, chondroitin sulfate 6-O-sulfotransferase and 4-O-sulfation-GalNAc-4-O-sulfotransferase.

3. The preparation method according to claim 2, wherein in Step (1), the chemical removal comprises: dissolving the K4 polysaccharide in an acid solution, heating to remove the fructosyl residue from glucuronic acid in K4 polysaccharide, and cooling to room temperature and dialyzing, to obtain DK4.

4. The preparation method according to claim 2, wherein in Step (2), the chondroitin sulfate degrading enzyme is chondroitin sulfate degrading enzyme ChAC, chondroitin sulfate degrading enzyme ChABC or hyaluronidase.

5. The preparation method according to claim 2, wherein in Step (2), the degradation comprises the following steps: dissolving DK4 in an enzymolysis buffer; adding the chondroitin sulfate degrading enzyme and reacting; and after the reaction, heating, undergoing solid-liquid separation, and collecting the filtrate, to obtain the chondroitin oligosaccharide mixture.

6. The preparation method according to claim 2, wherein in Step (2), the separation and purification of the chondroitin oligosaccharide mixture comprise the following steps: (1) subjecting the reaction solution to 30 kDa, 10 kDa, 3 kDa, and 1 kDa ultrafiltration and centrifugation sequentially, and collecting the supernatant; (2) separating the supernatant by chromatography on Bio-Gel P-2 gel, and collecting the eluate; and (3) collecting the eluate, detecting by HPLC, and collecting the analyte at a wavelength of 232 nm, to obtain the non-animal chondroitin oligosaccharide.

7. The preparation method according to claim 6, wherein the weight ratio of the chondroitin sulfate degrading enzyme to DK4 is 0.2:12-1:12; and the enzymolysis time is 10 min-24 hrs.

8. The preparation method according to claim 2, wherein in Step (3), the chondroitin oligosaccharide, the chondroitin sulfate sulfotransferase, and the sulfate donor 3′-phosphoadenosine-5′-phosphosulfate are mixed and reacted in a buffer, and the reaction solution is purified after the reaction, to obtain the non-animal chondroitin sulfate oligosaccharide.

9. The preparation method according to claim 8, wherein the molar ratio of the sulfate donor 3′-phosphoadenosine-5′-phosphosulfate to the chondroitin oligosaccharide is 4:1-0.5:1.

10. The preparation method according to claim 8, wherein the molar ratio of the chondroitin oligosaccharide to the chondroitin sulfate sulfotransferase is 1:0.1-1:20.

11. Use of the non-animal chondroitin sulfate oligosaccharide according to claim 1 in the preparation of drugs for treating nervous system diseases.

12. Use of the non-animal chondroitin sulfate oligosaccharide according to claim 1 in the differentiation of oligodendrocyte precursor cells in vitro.

13. The use according to claim 12, wherein the differentiation is to induce oligodendrocyte precursor cells to differentiate into oligodendrocytes.
